(19) United States
(12) Patent Application Publication
(10) Pub. No.: US 2005/0154434 A1
(43) Pub. Date: Jul. 14, 2005

(54) Clinical syringe with electrical stimulation aspects

(76) Inventors: Adam Simon, Yardley, PA (US); Arthur L. Lifshey, East Brunswick, NJ (US); Elena Fattori, Genzano (IT)

Correspondence Address: DUANE MORRIS, LLP;IP DEPARTMENT, ONE LIBERTY PLACE, PHILADELPHIA, PA, 19103-7396, US

(21) Appl. No.: 10/504,830

(22) PCT Filed: Mar. 6, 2003
(86) PCT No.: PCT/US03/06852

Related U.S. Application Data
(60) Provisional application No. 60/362,533, filed on Mar. 7, 2002.

(51) Int. Cl. A61N 1/30; A61N 1/00; A61N 1/05

(52) U.S. Cl. 607/116; 604/21

(57) ABSTRACT

A treatment device is provided for applying electrical energy to biological tissue in conjunction with injecting a composition that advantageously diffuses through the tissue. An injector such as a syringe has a reservoir for the treatment composition and injection cannula. The cannula can function as an electrode, and one or more additional electrodes are provided as opposed electrodes. The electrodes can each have tissue penetrating needles, or one or more of the electrodes can have a surface bearing conductive contact part. The syringe or other injector and a drive unit that applies electrical power to the electrodes are operable simultaneously or in a sequence, and can be triggered by switches or automatically upon detection of the injection proceeding to a predetermined state. The treatment device preferably uses a disposable syringe received in a carrier and generally is provided with a non-threatening appearance by minimizing tissue penetration and potential high voltage aspects.

### BACKGROUND OF THE INVENTION

1. Field of the Invention

The invention concerns a syringe apparatus for subsurface injection of compositions into biological tissues, together with simultaneous or post-injection application of an electric field to the site of the injection. For example, liquid medicinal compositions are injected into the muscular tissue of humans or animals using the apparatus, and the effects of the injection are modified (preferably optimized or amplified) by action of the electric field on either or both of the molecules of the composition and the tissue cells at the site of the injection.

The syringe apparatus is disclosed in embodiments suitable for day-to-day clinical use for injection and electrical stimulation of tissues. The apparatus is useful to facilitate infusion of injected pharmaceutical compositions into cells, for example to obtain or enhance an immunological reaction to the composition or its by-products.

2. Prior Art

It is known that applied electrical stimulation can affect biological tissues. A sufficient application of electromagnetic energy, for example, can increase the permeability of a membrane. This has possible therapeutic applications. Controlling membrane permeability, and in particular increasing permeability, may be useful when it is desired to permit solutions to diffuse through a membrane. It may be possible to affect the mobility of free ions in a solution by electrostatic effects. Applied electrical fields can affect a rate of diffusion through tissues by advection, or may vary the extent to which fluids diffuse into certain parts of the tissues. For example, electrical stimulation can increase permeability of a membrane when it is desired to infuse tissue with a substance through the membrane, and the rate of diffusion is at least partly a function of permeability.

Certain electrical or electromagnetic stimulation effects have been explained with reference to iontophoresis, electrophoresis and electroporation. These terms involve different forms electrical effects. They may be considered different ways to interpret the results that are caused by a given electrical potential, current or electromagnetic field. Depending on amplitude, polarity, frequency, spatial geometry and other parameters, a given field may produce a combination of such effects.

Iontophoresis and electrophoresis generally concern applying a direct current electric field in order to drive migration of positive and negative ions by electrostatic attraction and repulsion toward and away from an anode and cathode. Electric fields also tend to increase the mobility of the ions generally. Iontophoresis typically involves causing polar ions in a solution to migrate through an intact membrane such as the skin. Electrophoresis concerns the migration of ions in a fluid or gel under the influence of a polar electric field (i.e., a field with at least a direct current component).

Electroporation often involves a relatively higher power electric field, often applied briefly or pulsed. A field applied at sufficient amplitude and/or for a sufficient duration can induce microscopic pores to form in a membrane. The pores are commonly called “electropores” and the process of forming them is called electroporation. Depending on the power and duration of the energy applied to a membrane, the pores may be larger or smaller and may persist for a longer or shorter time. Preferably the pores persist temporarily, such as only during application of the field, and close or heal quickly. It is possible to damage tissue permanently by application of electromagnetic energy of too high a power level. Such damage might be caused by application of too high an energy level to a large volume of tissue such as a limb or other anatomical structure. The damage otherwise might be caused by application of a relatively smaller total energy level but wherein the energy is concentrated on a small volume of tissue (i.e., too high an energy density). Damage from electrical influence may produce untenably large or numerous pores such that the membrane fails to provide necessary containment. Electrical voltage and current may produce sufficient resistive heating that necessary biological processes can no longer be sustained. The electrical voltage and current also may have other effects on biological, chemical or physical processes.

In this disclosure the term “electrical stimulation” is not limited to any one or any particular combination of iontophoresis, electroporation, electrophoresis or any other electrical effects. The term as used herein is intended to encompass any such effects. A given electrical stimulation could have results that fall into more than one class, or possibly could be stronger in one or another due to the amplitude, polarity, spatial geometry and/or timing involved. For example, a given direct current or low frequency field could conceivably have sufficient amplitude to induce pore formation (electroporation) while also causing electrostatically driven ion migration through a membrane (iontophoresis) and accumulated migration with time (electrophoresis). Typically, however, electroporation involves higher electric field amplitudes than the other effects, and typically application at such amplitudes is brief or intermittent or is pulsed at a duty cycle that is sufficiently low to prevent unacceptable tissue damage.

The application of an electromagnetic field to tissue is complicated by the fact that tissue is not homogeneous, isotropic or otherwise regular from an electromagnetic perspective. An applied field and an induced current can become concentrated by variations in the material properties of the tissue, Including but not limited to the magnetic permeability and resistivity of tissues on a microscopic scale, and on a more macroscopic scale, by the anatomical structure and organization of tissue.

Electrically induced pores have been observed and studied to a degree, in vitro, where cells in a solution are substantially independent of one another and are exposed to view. It is difficult or impossible to observe the effects at a particular site in vivo. For example, obtaining access to tissue in vivo, such as sectioning the tissue to expose a site to view, tends to disturb the tissue in ways that alter the local amplitude, orientation or other aspects of the applied electrical energy. Thus it is difficult to make a meaningful in vivo observation of electrical stimulation parameters and effects.

Genetic and immunological therapies are candidates for electrical stimulation of tissues. Inasmuch as electrical stimulation tends to involve movement of ions and the opening of pores in tissues, it is plausible to apply a medicinal or other composition to a tissue site and to use electrical stimulation to move ions or molecules of the composition into positions, perhaps through pores in tissue membranes, where a desired effect is achieved or enhanced. Diffusion from thermal effects (Brownian motion) could drive diffusion through electroporated tissue membranes into an internal volume. Electrostatic or other electromagnetic effects could drive diffusion of ions through biological structures, or at least increase the motion of affected molecules (e.g., assuming an alternating polarity field), and thus affect particular reactions in order to achieve or to induce a therapeutic effect.

One such effect is the production of antibodies and potentially the stimulation of systemic production of such antibodies, as a means to induce or improve immunological attack on adverse pathologies such as viruses, cancer, antibiotic resistant bacteria, parasitic infections and other pathologies. Another such effect is to induce a strong antigen-specific cellular immune response.

U.S. Pat. No. 6,110,161—Mathiesen et al. (see also Mathiesen, 1999, Gene Therapy 6: 508_{—}514) discloses in vivo electrical stimulation of skeletal muscle within a calculated electric field strength ranging from about 25 V/cm to about 250 V/cm. WO 99/01158, WO 99/01157 and WO 99/01175 disclose the use of low voltage for a long duration to promote in vivo electrical stimulation of naked DNA. An electric field strength or voltage gradient of about 1 V/cm to about 600 V/cm is disclosed. U.S. Pat. Nos. 5,810,762; 5,704,908; 5,702,359; 5,676,646, 5,545,130; 5,507,724; 5,501,662; 5,439,440; and 5,273,525, disclose electroporation or electrical stimulation methods and apparatus that suggest useful electrical field strengths range from about 200 V/cm to about 20 KV/cm. U.S. Pat. Nos. 5,968,006 and 5,869,326 suggest electric field strengths as low as 100 V/cm for certain in vivo electrical stimulation procedures. These disclosures cover orders of magnitude in the intensity of the proposed voltage gradient. It is well known from the literature that the electrical properties of tissue are substantially resistive. Under Ohm's law, current dissipation is equal to voltage divided by resistance. Power dissipation or joule heating is proportional to the product of the voltage and current. Therefore, according to the disclosures and the wide ranges of proposed voltages, at least tissue heating, and possibly also other effects will vary substantially as well.

Jaroszeski et al. (1999, Advanced Drug Delivery Reviews 35: 131_{—}137), reviews in vivo electrically mediated gene delivery techniques. Titomirov et al. (1991, Biochem Biophys Acta 1088: 131_{—}134) discusses subcutaneous delivery of two plasmid DNA constructs followed by electrical stimulation of skin folds, using an electric field strength from 400 V/cm to 600 V/cm. Heller et al. (1996, FEBS Letters 389: 225_{—}228) discloses applying plasmid DNA expressing two reporter genes to rat liver tissue, including by generation of high voltage pulses (11.5 KV/cm) that were rotated in orientation using a circular array of paired electrodes.

These and other electrical stimulation studies are promising. They suggest that a limitation on some immunological techniques has been the fact that plasmids or other compositions introduced to invoke an immune response or the like, may not have been conveyed to their optimal location within the cell, and further that electrical stimulation might provide a way to improve the extent to which the compositions are placed where they will most dependably achieve the desired effect.

However, there are difficult challenges facing those who seek to apply the subject matter of such preliminary studies. Specifically, methods and apparatus are needed that are practical for administration, and that will be acceptable to patients and clinicians.

These problems are partly due to perceptions and are partly due to reasonable fears. Questions arise concerning the danger of pain, inadvertent shock and injury due to an electrical medical device delivering energy directly to the subject. There may be a fear of pain or injury associated with piercing of tissues, potentially if the associated device appears frightening as compared to a hypodermic needle. It may be particularly problematic if comparison with a hypodermic needle is unfavorable as to the size or number of tissue piercing parts, its association with unfamiliar and apparently-high-powered electrical apparatus and the like. There are also issues common to other therapy situations such as the sterility of implements that may be only partly disposable, possible expense, applicability to patients of different sizes and dispositions (e.g., children versus adults), etc.

For example, there is a reasonable perception on the part of many patients and physicians that electromagnetic energy can be dangerous. Some fears of electricity are controversial, such as the fear of long term damage from exposure to non-ionizing electromagnetic radiation from power lines. Other fears are from effects that are demonstrably real. For example, most people have had one or more experiences with startling and possibly painful electrical shock. Shocks from discharge of static electricity are common. An injurious or lethal electrical shock is possible at domestic voltage levels (e.g., 110 VAC), assuming good conductive connections. Many people can recall an experience with electrical shock from malfunctioning equipment. Sometimes a shock is caused by ignorance or error in making electrical connections. Sometimes a shock is due in part to deficiencies in product design. In general, most people are at least mildly suspicious of unfamiliar electrically-powered equipment, and also of the skill or attention of persons who operate such equipment.

A prospective patient may well hesitate if offered a therapy that involves attaching his or her person to a device that is coupled to the domestic electric power mains. In such apparatus, and even more so in apparatus that uses kilovolt pulses, safety precautions are essential and some design features are required by law. Precautions may include thickly insulated wires, grounding wires, high voltage insulation on leads and electrodes and the like. The appearance of such structures can aggravate a subject's fears.

Furthermore, at the voltage levels discussed above, such a patient's fears might be justified. An applied voltage of the magnitude discussed could produce a painful shock. If the voltage is less than painful, it may nevertheless cause muscle twitch or contraction or otherwise be disconcerting, uncomfortable or unfamiliar. Assuming that a procedure requires a certain voltage-to-distance ratio (voltage gradient), a relatively lower voltage could be applied using electrodes spaced more closely together. This focuses the electrical energy on a smaller area of tissue but does not prevent the physiological response of the tissue.

Some examples of electrodes intended to apply electromagnetic energy in conjunction with infusion of a substance by local or systemic injection, are shown in U.S. Pat. Nos. 5,439,440; 5,702,359; 6,009,347; and 6,014,584, all to Hofmann. U.S. Pat. No. 5,873,849—Bemard and U.S. Pat. No. 6,041,252—Walker et al. include disclosures of field patterns versus injection parameters, and discuss particular arrays of electrodes, for example in equilateral trios of anode/cathode arrangements in regular repetitive patterns of adjacent sets of electrodes that together encompass an extended area of tissue. Such an array of electrodes and associated electrical leads is rather impressive. Generally pulses are applied in a kilovolt voltage range using facilities that apply the voltage to the subject while protecting the technician from contact. The electrodes, leads, insulation and the like result in an electrical apparatus that is quite formidable in appearance.

U.S. Pat. No. 5,439,440, for example, teaches alternative contactors. In one arrangement a plier-like electrically insulated tool has paired-contactors that are mechanically arranged so as to compress or pinch a loose portion of flesh. In another arrangement, an array of ten needles forms the electrode structure of two spaced rows of five needles commonly. The two rows of five are coupled to the anode and cathode of a driving circuit, respectively. The '359 patent additionally discloses a circular array wherein needle-shaped contactors are connected by well-insulated connectors to a drive apparatus that resembles the ignition coil and distributor of an automobile engine. The '347 patent has a seven-by-seven position array of needle electrode positions. Each needle is mounted slidably in a contacting pad such that the advance of the needle can be controlled. The patent teaches positioning each needle in the array at a selected depth, for example with the distal ends or points of the needles being placed just inside the far boundary of a gland or other organ to be treated, and thereby placing a maximum length of each electrode in the tissue of the organ.

U.S. Pat. No. 5,674,267—Mir et al., teaches a similar arrangement comprising an array of needles. At least three are apparently used, but any number can be provided with each individual needle being paired with another needle. The device sequentially applies a driving voltage to each pair.

The contactors of the foregoing patents, which are hereby incorporated for their teachings of electrical arrangements and therapeutic methods, show that it is possible to place a number of electrodes into a volume of tissue so as to disperse the positions at which one or both terminals of an electrical circuit are coupled to the tissue. However the arrays of multiple needles may be reasonably frightening to the patient, particularly when combined protective design features that appear aimed at preventing inadvertent electrical shock. Such contactors are most useful in a situation in which the patient is not conscious or otherwise cannot see the needle array and electrical driving apparatus.

It would be advantageous to provide an apparatus that meets the needs for effective application of electrical power for therapeutic electrical stimulation applications, but to make the apparatus innocuous in appearance. It would also be advantageous if notwithstanding any de-emphasis on the electrical power and hypodermic injection aspects due to suitable tailoring of the visual appearance of the device, the device was capable of delivering a focused injection into a reasonably limited volume of tissue and to focus the application of electrical energy there. It would further be advantageous if all these facilities for electrical coupling and possibly piercing of tissues could be accomplished in a manner that is optimally safe for the technician, and reduces the danger of contact with body fluids, possible pricks from sharps, inadvertent shock and other associated hazards.

### SUMMARY OF THE INVENTION

It is an object of the invention to provide apparatus and methods for infusing a medical or pharmacological composition into tissue, in conjunction with application of electrical energy for interacting with the tissue and/or the composition. In particular, the invention is intended to provide a clinically optimal and practical device for simultaneously or sequentially effecting an injection and applying electromagnetic energy at the site of the injection.

Accordingly, injection and electrode structures are provided together. The injection aspects are arranged to be practical for the clinician and the electrical aspects are made tolerable for the patient or subject.

The injection structure preferably comprises certain elements that are substantially conventional, including a cannula for piercing the tissue, coupled to a collapsible volume for containing a liquid substance. Using additional electrodes and/or using at least part of the cannula and its carrying structure as an electrode and/or with a tissue surface contact electrode, an electromagnetic field is applied. Preferably, passive (manually operable) and/or active (automatic) movable portions are provided for protectively sheathing potentially injurious portions of the apparatus such as portions that contact tissues and/or have sharp points, for example deploying a protective cover, or operating to retract a dangerous structure.

Preferably the applied electric field is arranged to encompass substantially the same volume of tissue where the liquid substance is injected. The technician injects the substance and applies the field while the substance is present in the area. The field can be activated and the injection made in either order, provided that the field and the resulting electrical stimulation occur while the substance is infused in the area of interest. Preferably, automatic triggering and timing are employed to activate the electric field simultaneously with injection or for a period commencing after injection, and may also be used for automatic sheathing or automatic retraction of the cannula and/or electrodes.

The invention is practical and convenient. No expert skills are needed to arrange a field that will intersect the site of an injection, and to appropriately time the injection versus application of the field. Nevertheless, the arrangement is versatile in that it can be used with various treatment substances and to obtain various electrical field properties with respect to current, voltage and timing. In a preferred arrangement, the injector comprises a plunger and a sensor detects passage of a portion of the plunger so as to trigger the application of electrical energy simultaneously with passage or at predetermined later time or for a predetermined time interval.

These and other objects and aspects are met in the invention by a treatment device for applying electrical energy to biological tissue in conjunction with injecting a composition that advantageously diffuses or advects through tissue. The composition can diffuse through pores that are opened by the electrical energy or can involve other methods or effects of electrical stimulation. An injector such as a syringe has a reservoir for the treatment composition and injection cannula. The cannula can function as an electrode, and one or more additional electrodes are provided as opposed electrodes. The electrodes can each have tissue penetrating needles, or one or more electrodes can have a surface bearing conductive contact part. The syringe or other injector and a drive unit that applies electrical power to the electrodes are operable in a sequence, and can be triggered by switches or automatically upon detection of the injection proceeding to a predetermined state. The treatment device preferably uses a disposable syringe received in a carrier and generally is provided with a non-threatening appearance by minimizing tissue penetration and potential high voltage aspects.

A preferred embodiment is robust and comprises electrically reusable parts that are coupleable to the injector. The patient contact portions and the substance reservoir are disposable and inexpensive. The injector is meek in appearance, preferably minimizing the appearance of arrangements such as arrays of needle-like electrodes and injectors as well as aspects that suggest a painful or dangerous form of electrical energy.

### BRIEF DESCRIPTION OF THE DRAWINGS

There are shown in the drawings certain examples and embodiments of the invention as presently preferred, which are intended to illustrate aspects of the invention and not to limit the invention to the specific examples shown. Throughout the drawings, the same reference numbers identify corresponding or functionally equivalent parts.

FIG. 1 is a schematic illustration showing a clinical syringe device with electrical stimulation capabilities according to the invention, as deployed to treat tissue shown in section.

FIG. 2 is a detailed section view showing a treatment site and illustrating certain aspects of an alternative embodiment.

FIG. 3 is a corresponding section view showing an alternative embodiment with an associated triggering sensor.

FIGS. 4 and 5 are partial perspective views of the invention with alternative electrode arrangements.

FIG. 6 is a perspective view showing an embodiment comprising a carrier for receiving a disposable syringe or the like, and having multiple penetrating electrodes and mechanical contacts to make electrical connections.

FIG. 7 is a perspective view showing a combined cannula and electrode structure for use with a syringe barrel (not shown).

FIG. 8 is a perspective view showing a carrier for a syringe, with an alternative electrode assembly and a triggering sensor.

FIG. 9 is a perspective view showing an alternative penetrating electrode structure.

FIG. 10 is a perspective view of an alternative embodiment having a carrier with a spring biased electrode.

FIG. 11 is a perspective view showing an alternative embodiment and alternative resiliently biased electrode.

FIG. 12 is a perspective view for illustrating certain aspects of the respective embodiments.

FIG. 13 is a perspective view of an alternative embodiment similar to that of FIG. 5, shown partly in section.

FIG. 14 is a perspective view of another alternative embodiment, shown partly in section.

FIG. 15 is a section view along line 15-15 in FIG. 13.

FIG. 16 is a combined view showing a 1:1 electrode arrangement, or a removable-cannula 1:1:1 electrode:cannula:electrode arrangement, together with a table showing several possible polarity combinations therefor.

FIG. 17 is a plan view showing an alternative trilateral pattern of three electrodes and an optionally centered cannula.

FIG. 18 is a plan view showing an alternative quadrilateral or quincunx pattern of four electrodes with the cannula in the middle.

FIG. 19 is a schematic elevation view showing an exemplary lateral fluid emission pattern from two cannulae.

FIG. 20 is a schematic elevation view showing an exemplary axial fluid emission pattern.

FIG. 21 is an elevation view showing a practical embodiment using two disposable syringes in a carrier having electrical connections for using the syringe cannulae as opposed electrodes.

FIG. 22 is an elevation view corresponding to FIG. 1, wherein the syringes are carried on individual carriers that snap together with one another, and further wherein the syringe cannulae are diverted from a straight line configuration for adjusting the spacing between such electrodes.

FIG. 23 is a perspective view showing an alternative embodiment wherein a carrier has an on-board solid wire electrode and electrical connections for the electrode and for making contact with a syringe cannula fittable into the carrier, this cannula also being diverted for spacing purposes.

FIG. 24 is a perspective view of a carrier for a syringe (not shown) wherein the carrier has dual solid wire electrodes straddling a channel into which a syringe can be snap-engaged and operated.

FIG. 25 is a perspective view of a clasp for snapping over the syringe in the carrier to lock the syringe in place and also to make electrical connections as described below.

FIG. 26 illustrates an alternative configuration for making electrical contacts.

FIG. 27 illustrates an insert-twist plug member for use with a configuration as in FIG. 26.

### DETAILED DESCRIPTION OF PREFERRED EMBODIMENTS

For certain injectable pharmaceutical preparations it has been observed that electrical stimulation in the form of an applied current or electric field, at the site of the introduction of the pharmaceutical preparation, can possibly increase the effectiveness of the treatment compared to the same injection without the electrical stimulation. The present invention provides a mechanical and electrical means to provide the dosage form and the electrical stimulation simultaneously or sequentially with the same device during the same subcutaneous, intravenous, or intramuscular injection.

A treatment device 22 for this purpose, as shown generally in FIG. 1, can have a hypodermic syringe 32, and in addition to the sharpened cannula 34 of the syringe, has one or more additional electrodes 36. These can be penetrating electrodes of similar elongated gauge and sharpness as compared to the cannula 34 or can be shallower or deeper penetrating devices or even surface contactors. The additional electrode(s) 36 may be separate solid elongated metal “sharps” constructed from stainless steel or other conductive material. There may be one electrode, e.g., the cannula associated with the syringe, to be used with an opposed electrode that is otherwise coupled to the tissue to be treated, by any particular form of contacting device or non-contact radiating device.

According to a preferred arrangement, there are at least two electrodes associated with the syringe 32, or with a carrier or attachment for the syringe as discussed below. The cannula 34 can form one of the electrodes and any number of additional electrodes 36 may be provided to operate in opposition to one another or in opposition to the cannula as an electrode. The electrical signal can be applied in any timed sequence and/or spatial pattern of application of electrical energy of any polarity, amplitude or program of pulse, frequency, or amplitude modulation, for example as disclosed in the references mentioned in the foregoing Background of the Invention, which are incorporated in this disclosure.

The cannula of the syringe 34 is caused to pierce a biological tissue 50, such as human muscle tissue. As shown sectionally in FIG. 1, manual operation of the syringe 32, namely advance of a plunger 42 in the syringe barrel 44, discharges the contents of the barrel into the tissue 50 at a site 52 point below the tissue surface. According to the invention, an electrical signal is applied using at least two electrodes (one of which may be the cannula 34 and the other may be an additional penetrating electrode 36 as shown in FIG. 1). Alternatively, application of electromagnetic energy may be possible using electrodes that are otherwise spaced around the injection site 52, for example by an electromagnetic radiation technique. In any event, electromagnetic energy 55, indicated in FIG. 1 by a field represented by dash-dot lines, is applied over an area that at least partly encompasses the site 52 of the injection.

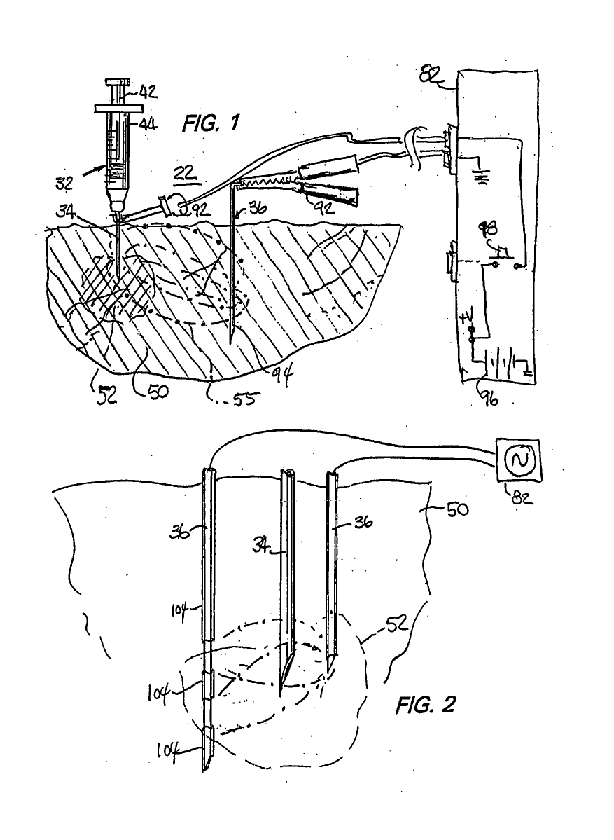

In FIG. 1, wherein the cannula 34 is used as one of the electrodes, the electromagnetic energy comprises a voltage gradient and resulting current flow that is directed toward the opposed electrode and thus part of the field may not intersect a large portion of the injection site 52. It is possible by providing electrodes that are spaced to straddle around at least part of the injection site, to cause the voltage gradient to intersect a larger part of the injection site 52, as shown in FIG. 2.

A number of specific arrangements are discussed herein, and the same reference numbers are used throughout the respective drawings to identify the same or functionally equivalent elements. In the embodiment of FIG. 1, the cannula 34 acts as one electrode or conducting pole of a pair 34, 36 carrying an electrical stimulation signal. Two electrodes 36 apart from the cannula 34 are shown in FIG. 2, etc. In some of the embodiments, two or more needles are parallel to and equidistant from a cannula 34, acting as opposed conductors or poles for the electrical signal, either together with one another or with the cannula. Both the cannula 34 and the sharpened penetrating electrodes 36 in that arrangement can be referred to as needles.

In another embodiment, the electrodes 36 apart from the cannula 34 are replaced by one or more surface contacting electrodes (not shown in FIGS. 1-3) that contact or possibly penetrate the skin or other tissue 50 less deeply, if at all, in the vicinity of the penetration of cannula 34. Several embodiments of such surface contacting electrode are disclosed, for example with reference to FIGS. 4, 5 and 8, comprising a range of materials and structures such as conducting sheet material (e.g., metal mesh, carbon coated or carbon containing polymer adhesive film electrode), resiliently biased contactors and the like.

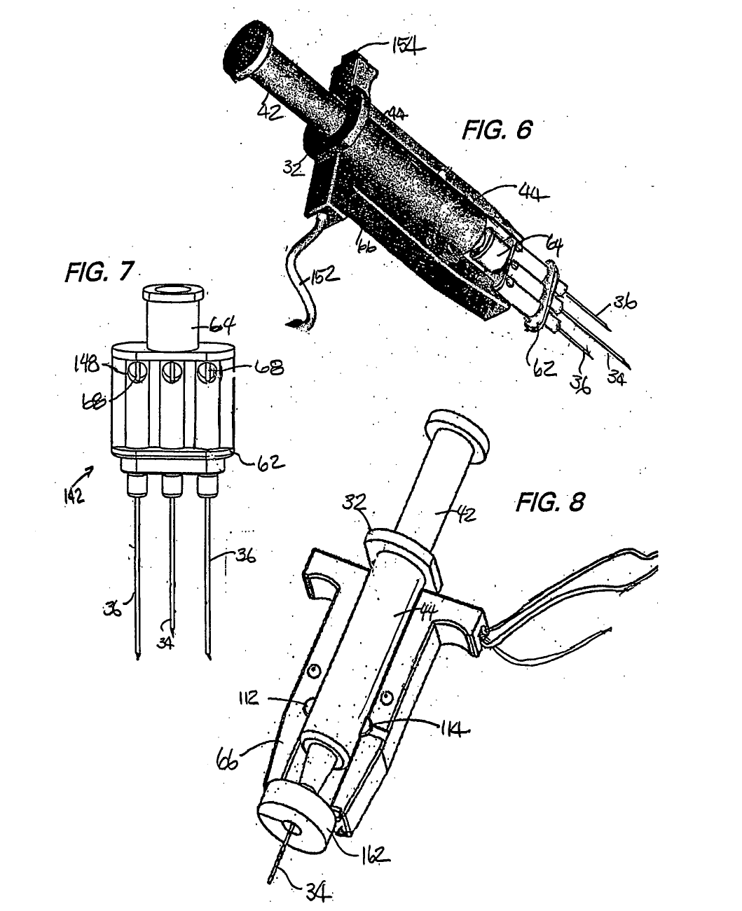

In certain preferred embodiments, a cannula 34 and one or more electrodes 36 are provided in a form apt for use with a disposable plastic syringe barrel. The cannula 34 can be commonly mounted with penetrating electrodes 36 on a plastic molded component 62, for example attachable to a disposable syringe barrel 44 using a standard Luer-lock fitting 64 as shown in FIGS. 6 and 7 or by other means (e.g., FIG. 9). Alternatively, a complete disposable syringe having a barrel 44, plunger 42 and cannula 34 can be received in a carrier 66 as in FIGS. 8 and 10-12. Electrical conductors can be provided on the electrode carrying component 62 or the carrier 66, or molded into their structures. Alternatively, resilient conductive contacts can be appropriately placed for making electrical contact with penetrating needles (possibly including the cannula) or for making contact with exposed metal or conductive parts so as to couple the driving signal to electrodes that are in turn coupled to the tissue at and/or adjacent to the site 52 of the injection.

In the embodiments discussed, the electrodes employed (one of which may be a cannula) are typically coupled to the anode and cathode of the drive circuit using conductors (i.e., wires). It will be appreciated that it is also possible to induce a current in a conductive electrode by a current induction technique. In that case an electrode or a conductor coupled to the electrode is irradiated with an alternating current electromagnetic signal to induce a current in the electrode, and the induced current produces the electrical stimulation effects sought.

Facilities such as electrical contacts, terminals, plug receptacles and the like preferably are spaced or similarly isolated from the insertable or penetrating or tissue-contacting portions of the electrodes and cannula(e), preferably by structuring the electrode carrying member 62 (preferably including the cannula 34) so as to separate and/or place a barrier between sterile tissue-contact and non-sterile electrical-contact portions. In this way, the electrical connections can be completed without compromising the sterility of the needles prior to injection, and the carrier can be used safely for successive treatments without the need to completely sterilize the carrier with each use.

The needle assembly, including all penetrating and/or tissue contact components, preferably comprises or is associated with a protective cover such as a displaceable needle sheath (not shown) that covers the injectable or fluid-contact portions of the needles (cannula and electrode(s)) before and after injection and treatment. The entire needle assembly and assembled sheath can be individually sterile packaged, and optionally pre-loaded with the composition to be injected.

The electrical connections area 68, for example as shown in FIG. 7, and the tissue engaging or penetrating structures 34, 36, can be isolated from one another by a suitable barrier of glass, plastic, metal or the like. In one embodiment, the structure effectively forming a barrier between the tissue contact and electrical contact functional portions also serves to provide a mechanical maximum limit or stop defining the depth of penetration of the needle(s).

It is an aspect of the invention to provide a clinically optimized device. This has many associated considerations such as the cost of various features and their effectiveness in achieving the necessary steps of injecting the tissue and applying the required electrical field. Advantageously, a wholly different objective is to make the device appear unintimidating to the patient. Insofar as possible, the treatment experience should actually be unintimidating to the patient, that is not painful, startling or otherwise uncomfortable. However, this sort of intimidation is affected by perceptions. In a preferred arrangement, the syringe/needle assembly as described above is associated with electrical and electronic components that appear to be low voltage apparatus or can be plainly battery powered. According to another aspect the penetrating components are kept to the same number as in a more conventional injection, namely one.

According to another aspect, the electrical driving signal can be triggered from the syringe or carrier (collectively the “treatment device”). A pushbutton can be provided for manual actuation. A mechanical limit switch (not shown) can be operated by advance of the syringe plunger to a given point, or in another embodiment the advance of the syringe to a predetermined triggering point is detected optoelectrically. The signals developed in these and similar ways can be coupled through a separate driving unit 82 that produces the signal for applying the electrical field to the tissue site. The driving unit 82 also can be arranged to operate visible and/or audible indicators that determine or indicate phases of operation such as a “ready” condition, the start and/or completion of the stroke of the syringe plunger, commencement of application of the electrical drive signal, etc., completion of a treatment cycle, system short circuit or continuity fault detection warming, and so forth.

Referring again to FIG. 1, the invention comprises a cannula 34 coupled to a source of a liquid treatment composition. In this example, the cannula is part of a common manually operable hypodermic syringe 32 such as a disposable plastic syringe, a glass syringe or the like, wherein the cannula 34, which is intended to pierce the tissue 50, is coupled to a barrel 44 that is collapsible or expandable by displacement of a piston or plunger 42 sealed to the inner walls of the barrel. The invention is applicable to other forms of injection and treatment device, such as automatic (e.g., motorized) syringes and syringes or other injectors having collapsible reservoirs structured in other ways, or other forms of pumps (not shown).

At least one electrode 36 is coupled to a source 82 of an electrical signal. Preferably, the cannula 34 is used as one of the electrodes, but it is also possible that the cannula could be electrically uncoupled or floating and that other conductors are used as electrodes. In this example the cannula 34 is coupled by a spring contact lead 92 to one terminal of the driving unit 82 and a second solid needle 94 is used as an opposed electrode 36, coupled to the other terminal of the driving unit 82 by a spring clip lead, which in this instance is an alligator clip. It would also be possible to use two complete syringes (not shown), the cannulas 34 of which are coupled to the driving unit 82 as opposed electrodes.

The source or driving unit 82 for the electrical signal is shown only generally in FIG. 1. A voltage source 96 is provided, which preferably comprises an on-board battery as opposed to a formidable-appearing AC wall plug cord. The driving unit 82 can have various arrangements for controlling the driving signal. The driving unit may be arranged to control the application of energy according to user input or the driving unit may be preprogrammed. The control can involve choice of various amplitude and timing attributes or simply the selection of available predetermined control parameter or control setpoints to be met by a feedback control arrangement. The voltage or current amplitude and pulse characteristics can be selectable or the user can select characteristics. In addition to amplitude and polarity, the signals can have selectable AC and DC components. The voltage polarity can be reversible. The signals can vary with time, such as a certain number of trains of pulses with interspersed pauses. The controller also can vary the application of energy spatially, for example reversing orientation by changing polarities (assuming two contact points) or by changing the orientation of the electric field in other ways, such as rotating the field direction be switching the signal to different pairs of electrodes in an array of three or more. In the embodiment illustrated schematically in FIG. 1, such switching functions are illustrated schematically by a simple switch 98. In a practical embodiment, the driving unit 82 typically comprises a power supply and one or more amplifiers or inverters that develop a voltage that is then applied by transistors or other switching elements to the electrodes. Provisions can be made in known manner for control and switching, such as polarity changes and the other variations discussed above. The driving unit 82 also can have safety features, for example to disable operation in the event of a detected short circuit, and adaptive features, such as controls operable to seek or maintain a preset parameter value (e.g., to control for a peak current setpoint), while varying other parameters (e.g., voltage) to achieve that value.

The syringe 32 is operated to discharge through the cannula 34, and the electrodes 34, 36 are electrically driven in a sequence that results in application of electrical energy to the subsurface tissue site 52 of the injection during the time that the injected composition is present. The electrical signal applied to the tissue site is sufficient to effect a stimulation response in the tissue at the site, or a reaction in the composition that is infused into the tissue.

There are different scenarios for precisely how electrical stimulation according to the invention can be used with therapeutic effects. Such effects naturally vary with variations in the electrical parameters, the infused composition and the tissue subjected to treatment. In one possible arrangement, the electrical signal could be sufficient to open pores in tissue membranes concurrently (or sequentially) with application of an injected composition. For example the composition could comprise plasmids that are to invoke immunological effects, and the pores might permit the composition to diffuse or advect more readily into the cells or into contact with structures subdivided by such membrane. It will be appreciated that in this and other ways, the combination of delivery of a therapeutic composition and application of an electric field may have an associated therapeutic effect or may enhance a therapeutic effect otherwise obtainable from the composition, but at a less vigorous rate. For example, the exposure of cells to plasmids in this manner may help to program the production of antibodies or may improve the robustness with which the tissue produces antibodies in response to a given quantity of composition.

The electrodes such as the cannula 34, and possibly other needles 36 used as electrodes, are electrically conductive over at least a portion of their surfaces in contact with the tissue. The needles can comprise an array of elongated needle structures extending substantially parallel to the cannula used for the injection. These needles are advantageously parts of a single treatment device, but can potentially be separate structures as in FIG. 1.

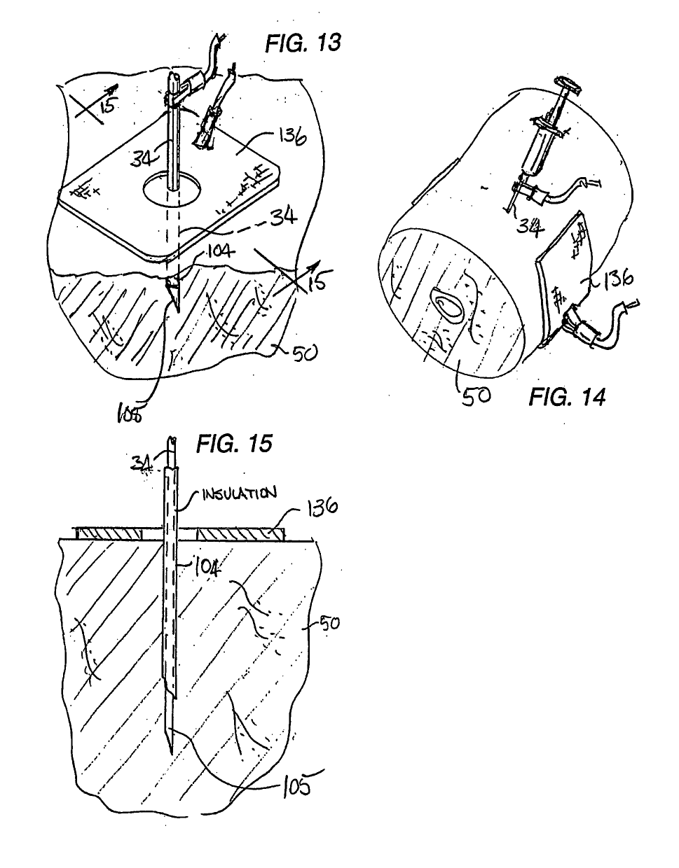

As shown in FIG. 2, the needles (cannulas 34 or other electrodes 36 used in the electrical circuit) need not be continuously conductive over their surfaces. On the contrary, it may be advantageous to provide an insulating or partly resistive coating over a portion of the otherwise conductive surface so as to concentrate the application of electrical energy to the site of the injection. Thus, for example, the electrode or cannula can have a substantially insulating plastic coating 104, for example of Teflon (polytetrafluoroethylene) over its length, except for a portion at the distal portion at the level of the injected composition (such an embodiment is shown and discussed with respect to FIGS. 13 and 15 below). FIG. 2 also demonstrates that the coating can be discontinuous to provide separated areas of concentrated application of electrical energy over the length of the needle (cannula or solid electrode or the like). In any event, the electrode provides a conductive surface in electrical contact with the tissue during penetration of the tissue by the cannula, or at least while the injected composition is present.

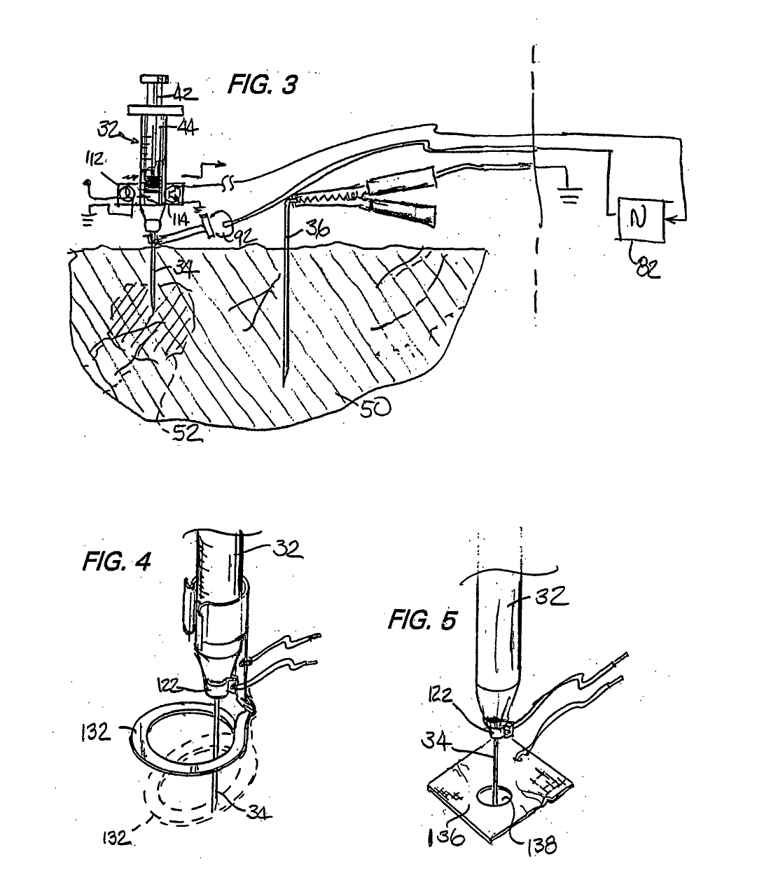

The signal applying electrical energy to the tissue is most useful if optimally spatially associated with the greatest application of the treatment composition to the tissue. FIG. 3 shows an embodiment in which the timing of the signal is specifically related to operation of the syringe to make the injection. In this embodiment (as in a number of the embodiments herein), the source of the treatment composition is a preferably-disposable plastic syringe 32 and the cannula 34 of the syringe functions as one of the electrodes driven by the electrical drive unit 82. At least one other electrode 36 is provided to make contact and the cannula and other electrode are coupled to the electrical drive unit as in FIG. 1. In the embodiment of FIG. 3, the drive unit 82 is triggered when sensing that the operation of the syringe 32 has advanced to the extent that there is expected to be a substantial concentration of the treatment composition in place at the site 52 of the injection. In the embodiment shown, the syringe barrel is substantially light transmissive (transparent or translucent). A lamp, LED or similar light source is provided on one side of the syringe barrel 44 and a photodetector 114 such as a photo diode or photo transistor is coupled to the barrel on a diametrically opposite side. The photodetector responds to light from the light source until the plunger 42 of the syringe, which generally is opaque at least at the plunger seal end, passes between the light source 112 and the photodetector 114, producing a triggering signal that is applied to the source 82 of the electrical drive signal. The source 82 can have a current source, threshold detector, one shot timer, amplifier and any other combination of components (not shown) to produce a driving signal between the electrodes (in this case between the cannula and the separate electrode) triggered by operation of the syringe. The signal can be maintained for a given time after triggering or may persist only so long as the plunger obstructs a line of sight between the light source and the photodetector, etc.

In FIG. 3, the electrical drive signal coupled to the cannula 34 is connected by spring clip 92 at the proximal end of the cannula adjacent to the syringe barrel 44. This connection point is available unless the cannula is to be inserted into the tissue to the hub at which the cannula is mounted. It is advantageous as previously discussed if the cannula is fixed to a needle assembly that has some form of sterile barrier located between the distal or injection end of the cannula 34 and an electrical contact area of the cannula, so that any concerns regarding the sterility of the electrical portions, which advantageously are intended to be reused, do not compromise the sterility of the injection.

FIGS. 4 and 5 demonstrate a contact electrode arrangement that is less intrusive than the penetrating electrode arrangements of FIGS. 1-3, but is still based on the use of a conventional preferably-disposable syringe and cannula. In FIG. 4, the cannula 34 is electrically connected to function as one of the electrodes via a clamp or spring clip collar 122 at its proximal end. The opposed electrode is provided by a sheet metal spring member 132 that attaches to the body of the syringe 32 and bears resiliently against the surface of the tissue 50 when the cannula 34 is inserted into the tissue to accomplish the injection. The spring member 132 can have an unloaded position, shown in dashed lines, that is relatively forward in the direction of injection, and can be deflected resiliently against pressure from the surface of the tissue (not shown in FIG. 4), such that the spring member 132 bends back into the position shown in solid lines. This places the electrode formed by spring member 132 at a predetermined relative position relative to the cannula 34, achieves electrical connection with the tissue by conductive contact, and also at least roughly determines the depth of the injection. A similar result can be obtained by providing a spring member 132 that is resiliently pivoted, for example being canted off the longitudinal center line at rest, and mounted such that pressure against the tissue during injection brings the spring member into a predetermined position when the cannula 34 reaches a predetermined injection depth.

Electrical contact between the tissue and the conducting parts of the arrangement shown can be facilitated by choosing the material on the surface of the electrode or its surface configuration. A conducting gel can be applied to improve contact. The surface can be smooth to increase the total surface area in contact or can be rough to increase the intimacy of electrical connection at discrete points on the surface. Similarly, the surface electrode need not be limited to surface contact and can have penetrating structures such as relatively short point contacts which compress and may pierce the tissue to a depth of up to a millimeter or so.

In the case of a surface electrode 132, the electric field in the tissue has a voltage gradient between the cannula and the spring member surrounding the cannula at the surface of the tissue, which is largely in a radial direction parallel to the tissue surface. The effectiveness of this arrangement for electrical stimulation may vary substantially with the depth of the injections or injections. Specifically, a deeper injection site may not be in optimal position to be subjected to a substantially radial voltage gradient at the surface, as compared to a shallower injection. In order to produce a voltage gradient that encompasses the site 52 of the injection (see FIGS. 1-3), the proximal part of the cannula in FIG. 4 can be insulated and only the distal end made electrically conductive at its surface for coupling with the tissue. An insulated arrangement is discussed below with reference to FIGS. 13 and 15. Insulating a length of the cannula provides an axial component to the voltage gradient and improves the extent to which the injection site is exposed to the electrical stimulation field.

FIG. 5 shows a related embodiment. In this case a surface electrode coupled to the drive unit 82 is not mounted mechanically on the syringe and does not bear against the tissue with resilient pressure as in FIG. 4. Instead, the surface contact comprises a flexible conductive sheet 136 with an opening 138. The injection is made through the opening 138 after attaching the sheet to the tissue. The sheet 138 can comprise a conductive mesh, a conductive plastic wherein carbon or other conductive particulate material is provided on the surface or throughout the material, a metal foil, etc. The sheet can be attached adhesively or using a conductive gel. As in the embodiment of FIG. 4, this arrangement is most effective with shallow injections or with deeper injections made using an opposed electrode, such as the cannula 34, which is insulated along its proximal length.

In the foregoing explanation, it is assumed that the cannula 34 discharges axially at its sharp end. It will be appreciated that the cannula 34 can be made to discharge laterally by providing a lateral opening or openings at a space from the distal end, which distal end also may be blocked. This method permits a portion of the cannula 34 to extend above and below the site of the injection. Such an embodiment is discussed below with reference to FIG. 19.

FIGS. 6-12 demonstrate several exemplary practical arrangements for the treatment device of the invention. These arrangements preferably are based in part on a standard syringe barrel 44 and plunger 42, but also are applicable to less standard injection apparatus. In FIGS. 8-12, the device can be used with a disposable syringe having a standard cannula 34. FIG. 6 demonstrates an embodiment in which the syringe barrel 44 is coupled to a delivery assembly 142, also shown in FIG. 7, comprising a central cannula 34 and a pair of needle electrodes 36 in a single unit.

Each of the embodiments shown in FIGS. 6-11 is a useful treatment device for electrical stimulation treatments and similar treatments in which electric energy is to be applied to tissue in conjunction with an injection. The drive unit 82 containing the electrical power supply and switching device operable to develop an electrical signal is not shown in these figures (see FIGS. 1-3). The device comprises an injector 32 whereby a reservoir for holding a treatment composition is coupled to an injection cannula 34 and has at least one electrode 34, 36, 132, etc., to be coupled to a drive unit 82. The injector and the drive unit are operable in a sequence as above, to apply the treatment composition to a subsurface site 52 in the tissue 50 by injection and to apply the electrical signal to the tissue site for affecting a reaction at the site to the treatment composition.

FIGS. 6 and 7 demonstrate an embodiment in which three needles are mounted on a block or assembly 142 such that the needles are exposed at the distal side of the block, and the block forms a barrier between that area and more proximal contact points at which openings 148 electrical contact can be made with the needles, or at least with conductors 68 that are electrically coupled to the needles. For example, the assembly block 142 can define openings 148 at which short lengths of the needles are accessible to be engaged with a spring clip or alligator clip. Alternatively electrical contact screws, contact plug/receptacle arrangements or the like can be provided. In one arrangement, the conductors which electrically engage with the electrodes or needles are provided in the form of plugs and mating receptacles. In these embodiments, the electrical contact area is well clear of the portion of the needles that is to penetrate or otherwise contact the tissue. This reduces concern for sterilization of the electrical components associated with the treatment device.

In the embodiment of FIGS. 6 and 8, the central needle is a cannula 34 and the most proximal part of the block comprises a collar for attaching the assembly block 142 to a syringe barrel 44 in the same way that a regular cannula might be attached, such as a Luer lock fitting. The assembly block 142 is attached to the syringe body. The electrical connections between the driving unit 82 (not shown in FIGS. 6-8) are made with the needles and the injection is accomplished.

In FIGS. 6 and 8, a conventional syringe is received in a carrier 66 that facilitates certain of the electrical connections and signal requirements needed to effect a treatment procedure. The syringe 32 is received in the carrier 66, for example being snap fit into the carrier, at a position at which the electrical connections are made by contact or can readily be made by making necessary connections. The respective electrical conductors can be gathered commonly into a cable 152, for example emerging from carrier 66 at a proximal end finger tab as shown in FIGS. 6 and 8. In FIG. 8, separate conductors emerge.

As shown in FIG. 6, the opposite finger tab can contain a manually operable triggering switch pushbutton 154 whereby the operator can enable or trigger operation of the electrical drive signal from drive unit 82 when ready, i.e., after the injection has proceeded to a predetermined point. Alternatively or in addition to a pushbutton, and as best shown in FIG. 8, the carrier 66 can have transparencies over an arrangement of a light source 112 and photodetector 114 as discussed with reference to FIG. 3 above, or other automatic means for triggering the signal, or perhaps to operate a warning or to trigger application of the signal in conjunction with other required inputs.

In FIGS. 6 and 7, the needles can be pressed to penetrate the tissue up to the full depth of the needles. The needles need not be of identical length; however assuming distal discharge of a central cannula 34, the voltage gradient and resulting electrical stimulation may be most effective if applied using outer electrodes 36 that are somewhat longer than the cannula. The length can be such that the likely volume of the treatment site to which the injected composition expands immediately before and during application of the electrical stimulation voltage gradient, preferably without substantially exceeding the length of the electrodes 36. The voltage gradient can be applied between the outer electrodes (i.e., not using the cannula as an electrode) or between either and/or both of the outer electrodes and the cannula in an alternating or other timed manner.

In FIG. 8, the same sort of carrier 66 is provided with a contact end plate in the form of a cylindrical pad 162 having a central opening through which the cannula protrudes, forming one of the two electrodes. The contact end plate forms the other electrode. The end plate can be axially displaceable against resilient pressure from a spring (not shown). Alternatively, the end plate can comprise a flexible cover on a resilient body that is compressed during an injection. In any event, the contact plate bears against the surface of the tissue and provides one point of electrical contact.

FIG. 9 shows an alternative embodiment in which a substantially conventional syringe body has a cannula and two spaced parallel electrode needles 36 on either side of the cannula. The cannula and the electrodes make electrical contact with a small circuit card 172 that serves to establish electrical connections and also can provide the mechanical mounting for the needles 36. The circuit card 172 can have conductive lands that are soldered to the needles or can have spring clips that engage the needles (not shown). The leads for the driving unit (not shown in FIG. 9) can be permanently terminated at the circuit card, e.g., by soldering, or can be attached to contact points on the card using spring contact clips or plug and receptacle connections.

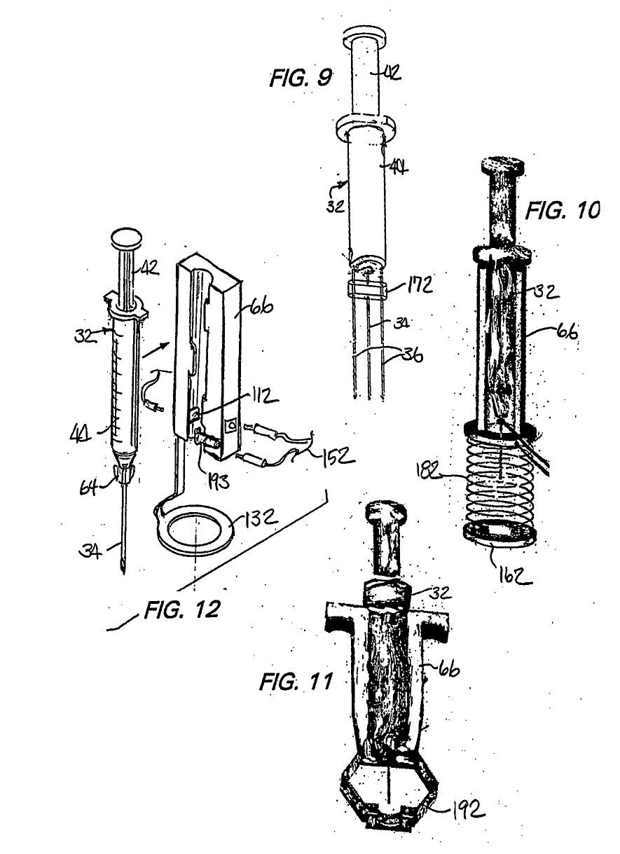

FIGS. 10 and 11 illustrate variations on the carrier arrangement of FIGS. 6-8. According to FIG. 10, the cannula 34 is one electrode and the opposed electrode comprises an annular contact plate 162 similar to that of FIG. 8, but bearing against the tissue under the resilient pressure of a helical spring 182. In FIG. 11, a similar arrangement has resiliently collapsible plastic arms 192 forming an enclosure with angled sides. As the device is used to make an injection, the distal side contacts the tissue and resiliently compresses the enclosure. This collapses the enclosure over a distance determined by the depth of the injection, while keeping resilient pressure between the distal side of the contact plate and the tissue.

FIG. 12 illustrates an example of a hybrid arrangement in which a conventional syringe such as a disposable plastic syringe 32 is snap-fit into a carrier 66 that comprises a light source and photodetector trigger generating circuit 112 as in FIG. 3, a spring like contactor as in FIG. 4 and a spring contact 193 for making electrical connection with the cannula 34. In addition to the electrical connections with the cannula as one electrode and the depending conductive contactor as the other electrode, plug and receptacle connections are provided for a connection cable 152. Preferably the syringe 32 snaps into the carrier at a predetermined position defined by the respective structures.

In the embodiments discussed, the carrier 66 has been coupled to an external electrical drive unit 82. In an alternative arrangement, a carrier as shown in FIGS. 6, 8, 11, 12, etc. can comprise a battery compartment and a compact circuit (not shown) to provide the driving signal for the electrodes. The driving signal may be a specific signal that is optimized for a given therapeutic treatment, which can be identified by appropriate labels, color coding or other identification of the carrier. The carrier can be supplied with pre-loaded syringes containing the required composition for the treatment for which the carrier's drive unit signal is adapted.

In FIG. 2 as discussed above, it is possible to employ electrodes that are electrically insulated by surface insulation 104 over a part of their length, so that the electric field gradient occurs substantially at the depth of an injected composition. FIGS. 13 and 14 apply a similar structural arrangement to an embodiment in which a surface electrode 136 forms one electrode, and a cannula bearing an insulating layer 104 forms the other electrode. The cannula comprises a conductive material such as stainless steel, but is insulated over a proximal portion of its length and thus makes electrical contact with tissue 50 only at the exposed conductive distal end portion 105 of the cannula. Thus the electric field gradient and resulting current are disposed between the distal end and the surface electrode 136, extending substantially axially.

Assuming that the cannula discharges in the area of its distal end, it is expected that the injected composition at least initially will occupy a volume that generally surrounds the point of the cannula. As a result, not all of the injected composition will be along a line between the surface electrode 136 and the conductive end 105 of the cannula. Insulating the proximal part of the cannula improves that application of the electric field to the part of the tissue where the composition is injected, but does not ensure that the field and the composition wholly coincide. According to an alternative method (not shown), an injection can be made to a given depth, followed by application of an electrical field at a corresponding depth, for example including insertion of an electrode or array of two or more electrodes after the injection, so as to provide a field that intersects at least some and preferably most of the tissue volume where the injected composition resides.

FIG. 14 shows an alternative embodiment in which a surface electrode 136 is used in opposition to an electrode/cannula 34 on opposite sides of tissue 50, for example on opposite sides of a patient's limb. In this embodiment the surface electrode 136 occupies most of the circumference of the limb and the injection is made to discharge at a depth, followed by electrical stimulation between the electrode/cannula at depth as generally surrounded by the surface electrode at the skin surface.

FIGS. 16-20 illustrate a number of array variations involving two or more electrodes or arrangements in which there are two or more electrodes of which optionally at least one, and potentially more than one, is a cannula having at least a portion that is conductive and is coupleable to a drive signal. In FIGS. 16-18, the cannula is shown in broken lines to indicate that the cannula can remain in place, for example being used as a electrode, or can be removable after making an injection, with other electrodes 36 being arranged to provide an electric field in the area of the injection. FIG. 16 shows that the cannula (assuming that it is left in place during electrical stimulation) can be Inert or powered. The polarities of the respective parts can be reversed and the signal can involve a potential difference between the electrodes or between the cannula and the electrodes, or between the cannula and either of the electrodes.

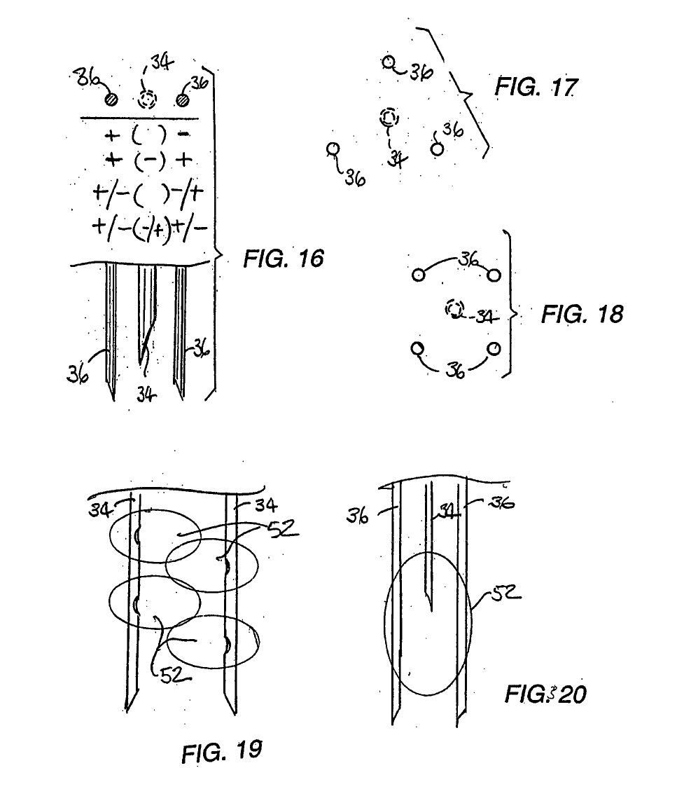

Similarly, the cannula can be employed with an array of three electrodes (FIG. 17) or four electrodes (FIG. 18) or more. In these arrangements the electrodes surround a centered cannula and can apply a field by connecting an electric potential in opposition to the cannula or in opposition to one or more of the other electrodes. In either case the effect is to apply a voltage gradient and to produce a current flow through the tissue in at least a part of the tissue that is exposed to the injected composition.

In FIGS. 16-18, the cannula and one or more electrodes are opposed conductors that apply the electrical energy. Preferably a plurality of opposed electrodes are placed at substantially equal distances from the cannula. In the respective embodiments, the electrodes and the cannula, or pairs or electrodes (or a subset selected from the electrodes of a larger array) function as a straddling pair, a triangle, a square, a pentagon, and a hexagon array of conductors.

FIG. 19 shows a two cannula arrangement in which the cannulae function as electrical connections and also inject the composition. In this embodiment each cannula is provided with one or more lateral openings, for example opening toward a space between the cannulae. The composition 52 is thus discharged at least substantially into the tissue area between the electrodes (cannulae). FIG. 20 shows an array in which one cannula 34 injects between two piercing electrodes. However, the cannula, which in this case discharges at its end, is shorter than the electrodes or at least is less deeply embedded in the tissue. As a result the field encompasses the area of the injected composition in an efficient manner.

The electrical stimulation treatment technique of the invention exposes the medical technician to some of the same dangers as occur with injections generally, such as exposure to potentially infected bodily fluids, and in particular exposure to possible infection introduced by inadvertent needle sticks. Inasmuch as the invention may employ piercing electrodes in addition to piercing cannulae, the danger of sharps injuries is multiplied. According to the invention, such dangers are minimized in several ways. As discussed, for example, with reference to FIGS. 4 and 5, one of the electrodes can be a surface contact member and the other can be the cannula itself, which reduces the number of sharp points involved. According to another arrangement, the sharp portions of the device are made retractable into a protective sheath to prevent pricks. According to a further arrangement, a deployable sheath extends to encompass the length of a cannula or electrode to prevent inadvertent contact with the sharp end. According to yet another embodiment, the deployment and retraction of the respective protective or dangerously sharp structures, respectively, are automated or timed in conjunction with the injection and the electrical stimulation steps.

Regarding retraction, U.S. Pat. Nos. 6,015,438 and 5,989,220—both to Shaw (Retractable Technologies, Inc., Elm, Tex.), the disclosures of which are hereby incorporated, teach structures whereby a sharp cannula can be engaged by a syringe plunger at the end of an injection stroke or a needle can be engaged when a sleeve cannula is pulled free, the engaged needle being retracted automatically into the syringe or cannula barrel by an axially directed helical spring. The present invention can employ a similar spring retraction structure. Preferably, according to the invention both the injection cannula and any piercing electrodes are arranged to retract, and this can be accomplished by a spring biasing arrangement as in Shaw. Similar arrangements for engaging and retracting needles into a safe and retracted position are disclosed in U.S. Pat. No. 6,156,013—Mahurkar; U.S. Pat. No. 6,090,077—Shaw; U.S. Pat. No. 6,096,005—Botich et al.; U.S. Pat. No. 6,099,500—Dysarz; U.S. Pat. No. 6,117,113—Novacek et al.; and U.S. Pat. No. 6,117,107—Chen. Alternatives that may include deployment of sheaths that enclose a sharp projection as opposed to retraction of the projection into a sheath (which actually involve substantially the same sort of relative motion) are disclosed in U.S. Pat. No. 6,162,197—Mohammad; U.S. Pat. No. 6,156,011—Ruminson; U.S. Pat. No. 6,149,629—Wilson et al.; and U.S. Pat. No. 6,156,012—Nathan.

It is an aspect of the present invention that retraction of an elongated sharp structure or deployment of a sheath to confine the sharp structure, can be triggered automatically by operations according to the invention. As discussed above, the electrical drive unit 82 can be triggered by a signal developed optoelectrically (or otherwise) when the plunger of a syringe reaches a particular point of advance. As a timing matter, the drive unit can be arranged to produce a signal at the conclusion of electrical stimulation that causes retraction of the cannula and/or the electrodes, or deploys a protective sheath.

In that embodiment, the cannula or electrode can be biased toward retraction by a compressed helical spring, for example as in Shaw '438, and held in an advanced position against spring pressure, in part by a fusible link structure coupled in a circuit with the drive unit. The fusible link is normally strong enough to provide a structural hold to keep the cannula or electrode in the advanced position against pressure of the compressed spring. At the conclusion of treatment, the drive unit couples a sufficient electrical current to the fusible link to melt the link, thus breaking the structural hold. The spring then retracts the cannula or elongated electrode into the body of the electrical stimulation device.

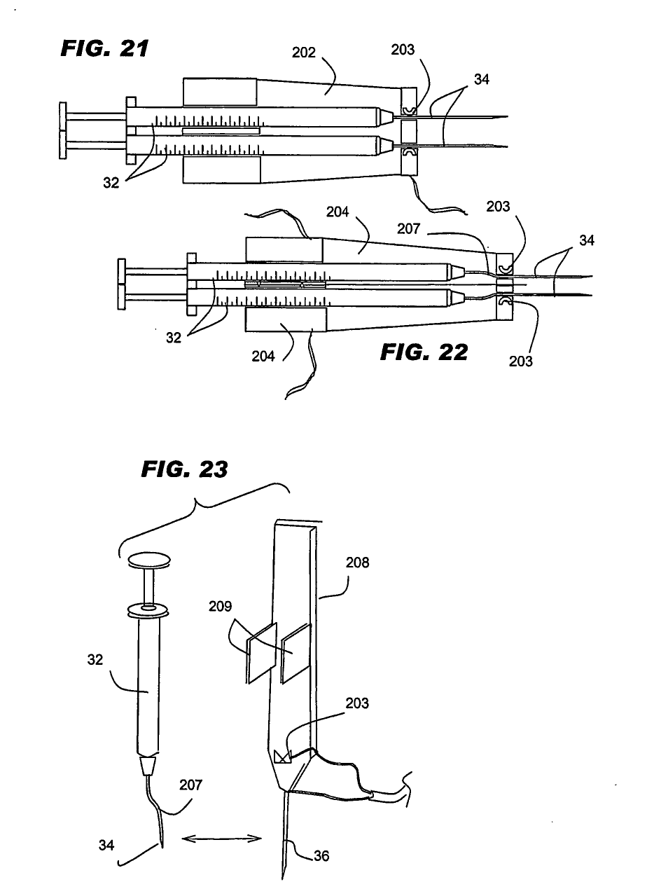

FIGS. 21-27 illustrate a number of practical applications of the invention, including particular structures for use with popular inexpensive syringes, particular carriers for such syringes, robust and dependable techniques for obtaining good electrical connections, and associated sensing devices whereby application of electrical energy can be triggered by a signal generated when the injection of the therapeutic agent reaches a predetermined point in its progress.

FIGS. 21-23 illustrate alternative syringe tube/cannula/electrode assemblies. These embodiments comprise carriers and syringes, which can by supplied individually in separate sterile packages, or as an optionally pre-assembled kit. Preferably the syringes used are conventional low cost disposables. It is also possible to employ a specially structured syringe, such as one in which portions of the cannula have electrically insulating surfaces.

In the embodiments shown in FIGS. 21-23, the cannula 34 of the syringe 32 functions as at least one of the electrodes. The devices comprise one or more suitable syringes 32 with attached cannulae 34, and means for making electrical contact as described above, preferably including structure for physically attaching the respective parts together securely, such as a snap-together arrangement.

FIGS. 21 and 22 show plural-syringe arrays. For example, a standard 1 cc tuberculin syringe 32 with an attached needle/electrode (i.e., cannula) 34 is mechanically snapped into a molded plastic holder 202. The holder fixes the position of the syringe and cannula during the injection, without interfering substantially with handling of the syringe barrel and plunger. The holder 202 also ensures electrical contact with the needle/electrode, preferably by use of one or more spring loaded metal contacts 203 for each needle. The spring contact 203 can be attached to electrical leads, or can be electrically connected to a convenient pin or plug contact (not shown) located clear of the needle and the plunger.

In FIG. 21, two standard syringes 32 are received in respective complementary depressions or channels in a single molded plastic carrier 202, each channel having an electrical contact structure engaging against the cannula or needle. The two syringes can be used simultaneously to inject the same or different components, and for this purpose it is possible to operate the plungers together (via an optional clip—not shown—for attaching the plungers to move as one) or separately, e.g., one after the other in a sequence or timing technique that also involves timing of the application of electrical energy.

FIG. 22 illustrates an alternative in which each of the syringes has its own discrete carrier 204. However the carriers 204 are attachable to one another by snap fitting pins and receptacles, shown schematically.

As also shown in FIG. 22, it is possible to divert the needle or cannula parts of standard syringes to adjust the spacing between two needles used as electrodes, at least one also being used as the injection cannula. The syringes can be specially manufactured with such a diversion or Z-bend 207 in the needle, allowing the needle to be closer to an adjacent needle that would otherwise be permitted if one or both of the respective needles was wholly straight and aligned to the longitudinal center axis of the syringe. In the case of a syringe manufactured specifically for use as shown, it is also advantageous to supply the syringe and carrier in a sterile assembly with the syringe pre-filled, volume adjusted and air-evacuated, etc. Alternatively, the technician can fill the syringe in a conventional way from a standard stoppered vial.

The electrical leads are coupled to a source for generating the electrical signal as discussed above (not shown in FIGS. 21-23). The embodiments shown are such that the electrical conductors can be placed so as to prevent or inadvertent shock by contact with conductors apart from the needle electrodes themselves.

In FIG. 22, the two snapped-together carriers 204 carry individual needles or cannulae 34. It would be possible to have more than two carriers that snap together in a similar array, using suitably complementary structures. However the embodiment shown is specific for two attached carriers.

FIG. 23 illustrates another arrangement wherein a carrier 208 receives a snap-mounted standard syringe and has a second electrode 36 that is not a syringe cannula. The carrier electrode 36 can be a solid sharpened wire of suitable strength. As in FIG. 22, the syringe cannula 34 can have a Z-bend 207 as shown to adjust the spacing between the electrodes. As an alternative, a Z-bend can be provided exclusively in the solid wire electrode of the carrier, which is shown as straight in the example of FIG. 23. It is preferable that only one of the electrodes be diverted if necessary to adjust the electrode spacing, and making the solid wire the diverted electrode, rather than using a Z-bend syringe, permits use of an unmodified standard syringe and cannula.

In FIG. 23, the electrical connection to the solid wire electrode can be made through a conductor molded into the carrier. The electrical connection to the syringe cannula is made through a metal spring clip 203 that clips over the cannula. The barrel of the syringe can snap resiliently between molded channel walls 209 as shown.

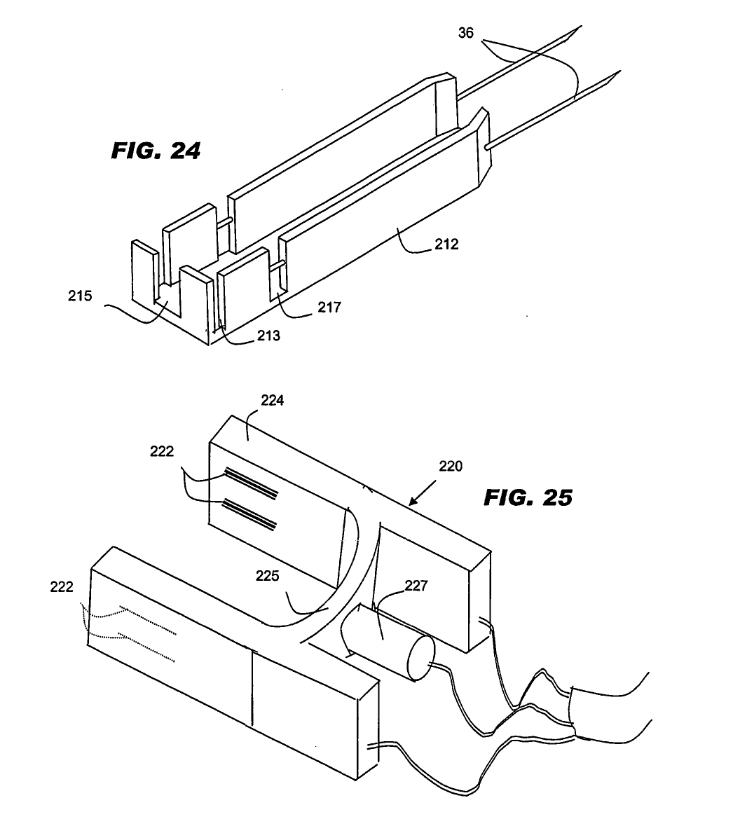

FIG. 24 shows an alternative embodiment that can be used with an unmodified standard syringe (not shown in FIG. 24). The carrier 212 in this case is formed by an integrally molded plastic channel member that has two solid wires extending through the molded material of the side walls to emerge as sharpened protruding electrodes 36. The channel member carrier 212 is dimensioned to snap around a standard syringe (not shown), the cannula of which syringe is to be straddled by the electrodes 36 molded into the walls of the channel member 212. The channel member is structured at the end opposite from the electrodes to engage with the upper end of the syringe barrel, having lateral openings 213 into which the usual finger tabs at the end of the barrel can be fitted. A longitudinal gap 215 provides clearance for the plunger of the syringe.

A preferred manner for coupling electrical signals to the device of FIG. 24 is shown in FIG. 25. As shown in FIG. 24, the electrode wires are exposed at two gaps 217 in the walls of the channel member. A connecting clip device 220, shown in FIG. 25 (in a larger scale than FIG. 24), engages with the electrode wires at the gaps 217 in the channel member carrier 212. The connecting clip 220 acts for securing the syringe barrel in the carrier 212, making electrical connections with the electrodes, and preferably also carrying a sensor to detect the injection status of the syringe plunger.

The clip device 220 has four electrical contacts 222, namely two contacts on each leg part 224 as shown. The leg parts 224 are coupled by a bridging part 225 at a midpoint along the leg parts and operate to clasp resiliently onto the electrode wires like a clothespin. By pressing together the upper ends of the legs 224, the lower ends are resiliently moved apart and when released on the contacts 222 clamp resiliently against the electrodes at the gap 217 to obtain good electrical contact.

Two spaced electrical contacts 222 are provided on each leg. Two coupled contacts on each leg could be provided for simple redundancy to ensure that the signal generating device is properly connected. However according to a preferred arrangement, two separate contacts are provided on each leg, coupled through their contact with the electrode. In this way, the control unit (discussed above) can sense whether the carrier is properly connected to the electrical signal generator by sensing for continuity between the contacts on each individual leg. The doubled contacts 222 can also independently verify that the electrical signal is properly coupled to the patients tissues, namely by monitoring both the current in the lines and the voltage across the lines. This arrangement ensures electrical connection and application of the required electric field.

The bridging part 225 of the connecting clip 220 carries a proximity sensor 227 that produces a signal developed as a function of passage of the syringe plunger at least to a predetermined point along an injection stroke. The point can be full injection or partial. Various specific kinds of sensors can be used to produce a signal based on electromagnetic, optical or sonic variations, etc. For example, the sensor can respond to a magnetic or reflective material on the plunger. The sensor can produce an analog level, a contact closure or a switched edge from a semiconductor switch element.

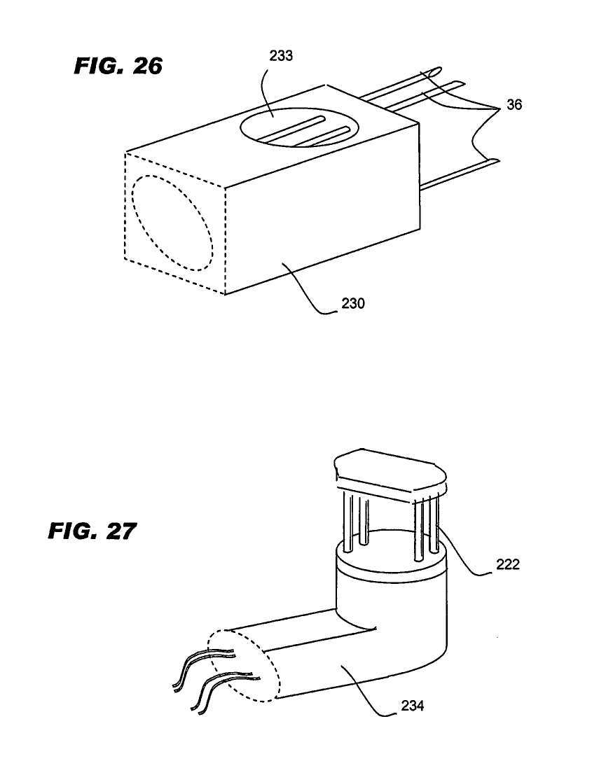

FIGS. 26 and 27 illustrate an alternative embodiment. As shown in FIG. 26, two (or more) electrodes 36 are spaced from one another and span an opening 233 in a molded plastic element 230, shown generally, for holding a syringe (not shown). A connector 234 as shown in FIG. 27 is inserted and turned by 90 degrees, such that two conductor posts 222 are pressed against each of the electrodes 36. In other respects, the connections can be handled as discussed above.

In light of this disclosure, a number of additional variations and embodiments will be apparent to persons skilled in the art. The invention is reasonably intended to encompass a range of variations in accordance with the foregoing disclosure and as defined in the appended claims.

1. An apparatus, comprising:
a cannula coupled to a source of a liquid treatment composition;
at least one electrode for use in a pair of electrodes coupled to a source of an electrical signal; and,
wherein the cannula and the electrode are operable in a sequence to deliver the treatment composition to a subsurface tissue site by injection and to apply the electrical signal to the tissue site for affecting a reaction at the site to the treatment composition.

2. The treatment apparatus of claim 1, wherein the cannula is electrically conductive over at least a portion of a surface thereof and functions as a further electrode.

3. The treatment apparatus of claim 1, wherein the electrode comprises an elongated needle structure substantially parallel to the cannula.

4. The treatment apparatus of claim 3, wherein at least one of the cannula and the elongated needle structure of the electrode is conductive over a limited portion of a surface thereof.

5. The treatment apparatus of claim 1, wherein the electrode comprises surface contact positioned to bear against the tissue during penetration of the tissue by the cannula.

6. The treatment apparatus of claim 1, wherein the cannula and the source of the treatment composition are portions of a disposable syringe unit.

7. The treatment apparatus of claim 6, wherein the cannula is fixed to a needle assembly comprising a barrier disposed between an injection end of the cannula and an electrical contact area of the cannula.

8. An electrical stimulation treatment apparatus for treating a tissue, comprising:
a drive unit containing an electrical power supply and switching device operable to develop an electrical signal;
an injector comprising a reservoir for a treatment composition coupled to an injection cannula and at least one electrode coupled to the drive unit, wherein the injector and the drive unit are operable in a sequence to apply the treatment composition to a subsurface site in the tissue by injection and to apply the electrical signal to the tissue site for affecting a reaction at the site to the treatment composition.

9. The apparatus of claim 8, wherein the drive unit is battery powered.

10. The apparatus of claim 8, wherein the injector has a movable part that is advanced to inject the composition and further comprising a sensor responsive to the movable part, the sensor being coupled to the drive unit for triggering the electrical signal at a predetermined position of the movable part.

11. The apparatus of claim 8, wherein the injector comprises a syringe tube and the movable part is a plunger advanced in the syringe tube for injecting the composition via the cannula.

12. The apparatus of claim 8, further comprising an alarm producing one of a visible and audible signal during application of the electrical signal.

13. The apparatus of claim 8, wherein the injection cannula acts as an electrode coupled to the drive unit in a circuit including the tissue.

14. The apparatus of claim 8, comprising at least two electrodes coupled to the drive unit, wherein the two electrodes and the tissue form a circuit with the drive unit during at least one of simultaneous and sequential injection using the cannula.

15. The apparatus of claim 14, wherein at least one of the electrodes comprises a spring biased contactor operable to bear against a surface of the tissue during the injection.

16. The apparatus of claim 15, wherein at least one of the electrodes comprises an elongated conductor that penetrates the tissue during the injection.

17. The apparatus of claim 16, wherein the elongated conductor that penetrates the tissue comprises the cannula.

18. The apparatus of claim 17, wherein the contactor at least partly surrounds a penetration point of the cannula in the tissue.

19. The apparatus of claim 8, wherein the injection cannula and the at least one electrode form a spatial array comprising electrical contacts that at least partly surround a volume at which the composition is injected.

20. The apparatus of claim 19, wherein the electrical contacts are formed by the cannula and at least two electrodes, the contacts straddling at least part of the volume at which the composition is injected.

21. The apparatus of claim 20, wherein the electrical contacts comprise a plurality of electrodes.

22. The apparatus of claim 8, wherein at least one of the cannula and the at least one electrode has a conductive surface over only a predetermined part of a surface exposed to the tissue.

23. An apparatus for delivering a pharmaceutical agent, comprising a cannula having a surface that is electrically conductive at least in a predetermined area, and at least one opposed electrode, wherein the cannula and the opposed electrode are positioned to at least partly bound a volume into which the cannula discharges.

24. The apparatus of claim 23, wherein the cannula is arranged to pierce tissue and the opposed electrode bears against a surface of the tissue.

25. The apparatus of claim 23, wherein the cannula and the opposed electrode are elongated structures for piercing tissue and are conductive at least on portions of surfaces contacting the tissue.

26. The apparatus of claim 25 comprising a plurality of opposed electrodes placed at substantially equal distances from the cannula.

27. The apparatus of claim 26, wherein the opposed electrodes form an array defining at least one of a straddling pair, a triangle, a square, a pentagon and a hexagon.

28. The apparatus of claim 27, wherein the cannula is substantially centered in the array.

29. The apparatus of claim 23, further comprising a mechanically driven retraction drive operable to withdraw at least one of the cannula and the electrode from the tissue.

30. The apparatus of claim 29, wherein the retraction drive comprises a spring biased toward retraction and a triggerable release.

31. The apparatus of claim 23, wherein further comprising a carrier for removably receiving the cannula.

32. The apparatus of claim 31, wherein the carrier comprises at least one electrode and at least one electrical conductor.

33. The apparatus of claim 32, wherein the carrier comprises a conductor coupleable to the cannula by contact, and a conductor coupled to the electrode.

34. The apparatus of claim 32, further comprising a connector operable to couple at least one of an electrode and a cannula to a signal generator, the connector being removably coupleable to the carrier.

35. The apparatus of claim 32, wherein the connector comprises at least two contacts that both are removably coupleable to one conductive element selected from the group consisting of the cannula, the electrode and the electrical conductor, and wherein the apparatus is responsive to continuity between said two contacts indicative of electrical connection with said conductive element.
